(19) United States
(12) Patent Application Publication
(10) Pub. No.: US 2010/0288291 A1
(43) Pub. Date: Nov. 18, 2010

(54) METHOD FOR PREVENTING OR TREATING PRESSURE SORES

(76) Inventor: Michelle A. Friedman, Los Angeles, CA (US)

Correspondence Address: Ira M. Siegel;LAW OFFICES OF IRA M. SIEGEL, Suite 970, 433 N. Camden Drive, Beverly Hills, CA, 90210, US

(21) Appl. No.: 12/467,252

(22) Filed: May 15, 2009

(51) Int. Cl. A61F 13/00

(52) U.S. Cl. 128/889

(57) ABSTRACT

The invention is a method for treating or preventing pressure sores. It comprises (i.e., includes but is not limited to) the following steps: applying a cushion in indirect contact or in direct contact with substantially the entire surface of a pressure sore area, and applying pressure against the pressure sore area through the cushion. The cushion is preferably circular in shape (although it may have other shapes) with its diameter between about 1.5 and about 6 times its height, and preferably between about 3 and about 4 times its height. In the preferred embodiment, the cushion has a silicone elastomeric shell, with the portion of the shell intended to be in occlusive contact with the pressure sore having a smooth outer surface. In the preferred embodiment, the cushion is only partially filled, and the fill material is silicone gel. The cushion may be held in place by means of a strap, wrap, bandage or similar device.

### BACKGROUND OF THE INVENTION

1. Field of the Invention

This invention relates to the field of wound care, prevention and treatment for pressure sores.

2. Prior Art

Pressure ulcers, pressure sores, decubitus ulcers, bed sores (herein referred to collectively as “pressure sores”) are the bane of the bedridden and the wheelchair bound, and of their caregivers. For those hospitalized people who would otherwise be on the road to recovery from an illness or injury, the onset of pressure sores can increase hospital stays by as much as 50%, or more. For those residing in nursing homes, pressure sores can complicate what should be a restful experience into a punishing stay. For those required to use a wheelchair to get around, pressure sores can add severe pain to the inconvenience already being suffered. All pressure sore sufferers are at an increased risk of infection. Pressure sores can even lead to sepsis and/or to early death.

Pressure sores are lesions which can start off about a half inch across and can become as large as the size of a palm of a hand, or even larger. Surface size is only one measure of the extent of a pressure sore. A pressure sore typically starts at the surface of the skin. If left untreated, over time (and sometimes that time is very short) a pressure sore can extend deep into muscle and/or bone.

Pressure sores are classified into stages. The stages are generally described as follows:
Stage 1: A lesion of the epidermis (outer surface of the skin) which is reddish in color on lighter-skinned people and purplish or bluish in color on darker-skinned people and which does not lighten when pressed. The lesion may be painful.
Stage 2: Superficial damage to the epidermis, with a blister or an abrasion.
Stage 3: The lesion extends to subcutaneous tissue.
Stage 4: The lesion extends to muscle or bone.

People with reduced ability to move are the most susceptible to pressure sores. It is believed that pressure sores are the result of continuous pressure and/or friction at a particular area of skin, particularly an area of skin close to bone or cartilage, such as the spine, knees, ankles, heels, back, hips, and coccyx (tailbone).

Estimates are that more than one million people suffer from pressure sores in America alone. The numbers of people who will be at risk of suffering pressure sores is likely to increase dramatically because (i) older people are more likely to be involuntarily immobile, (ii) the skin of people tends to thin as they age, and (iii) a large cohort of people are about to start leaving middle age.

Treatment of pressure sores with prior art methods, even if treatment starts when a pressure sore is in an early stage, usually requires weeks for a successful resolution to be obtained, if one can be obtained at all.

One basic measure for treatment and/or prevention of pressure sores requires frequent turning by a caregiver of an immobile patient. To some extent, a wheelchair bound person may be able to move potentially affected portions of his own body himself. The turning is recommended at least once every two hours. Each time the body is turned, pressure is at least temporarily relieved from the areas previously under pressure and allows them to be air-cooled.

Additional treatment measures include administering oral antibiotics, cleaning the pressure sore and the area around it, including removing any dead tissue, applying topical antibiotics and/or other salves and dressings such as available under David Dixon Dermawound brand, 3M's Tegaderm brand, Convatec Inc.'s DuoDerm brand, Coloplast's Contreet brand, and King Pharmaceuticals' Silvadene brand.

Dressings and salves do reduce friction and further skin breakdown, and they help reduce the risk that an affected area would be contaminated by the results of bowel and/or urinary incontinence suffered by some patients. However, healing still is uncertain and takes weeks.

Other measures for preventing and/or treating pressure sores include having the patient rest on an alternating pressure mattress and/or on an air mattress having an air-permeable surface. The purposes of such mattresses are to temporarily transfer pressure from the pressure sore to nearby parts of the body and to provide airflow to the pressure sore, which among other things, could allow for cooling of the affected tissue. Even when mattresses are efficacious, they are expensive to buy and expensive to operate and maintain.

To prevent or alleviate pressure sores, various cushions and pads have been used, such as those disclosed in U.S. Pat. No. 3,721,232. Some are made of foam or of gel, and some are filled with air or water. These cushions and pads have a body-contacting surface area which is intended to distribute the pressure which results from lying on a bed or sitting in a chair away from the pressure sore lesion to other parts of the body. Some of these pads and cushions are formed with a void, with the pressure sore to be positioned over the void. Some of the voids are formed by having the pad or cushion shaped as a donut or ring.

The efficacy of the cushions and pads used in the prior art treatments still leaves much to be desired.

### SUMMARY OF THE INVENTION

The invention is a method for treating or preventing pressure sores. It comprises (i.e., includes but is not limited to) the following steps: applying a cushion in indirect contact or in direct contact with substantially the entire surface of a pressure sore area, and applying pressure against the pressure sore area through the cushion. The cushion is preferably circular in shape (although it may have other shapes) with its diameter between about 1.5 and about 6 times its height, and preferably between about 3 and about 4 times its height. In the preferred embodiment, the cushion has a silicone elastomeric shell, with the portion of the shell intended to be in occlusive contact with the pressure sore having a smooth outer surface. In the preferred embodiment, the cushion is only partially filled, and the fill material is silicone gel. The cushion may be held in place by means of a strap, wrap, bandage or similar device.

In accordance with present invention, and contrary to prior art methods, increased pressure is applied locally to the pressure sore area.

### BRIEF DESCRIPTION OF THE DRAWINGS

FIG. 1 is a side elevational view of a patient rolled over on to the patient's side while lying on a mattress. The patient is shown having a pressure sore by the patient's coccyx.

FIG. 2 is a side elevational view of a patient rolled over on to the patient's side while lying on a mattress. The patient is shown having a pressure sore, shown in dashed lines, by the patient's coccyx, with a circular cushion used in the invented method applied in contact with the pressure sore.

FIG. 3 is a side elevational view of one embodiment of a cushion used in the invented method.

FIG. 4 is a side elevational view of another embodiment of a cushion used in the invented method.

FIG. 5 is a cross-sectional view of the embodiment of the cushion shown in FIG. 4.

FIG. 6 is a top plan view of the cushion shown in FIG. 4.

FIG. 7 is a bottom plan view of the cushion shown in FIG. 4.

FIG. 8 is a sectional view (not to scale) of a portion of a human body, which body has a pressure sore, resting on a portion of a mattress.

FIG. 9 is a sectional view (not to scale) of a portion of a human body, which body has a pressure sore, with a cushion disposed against the pressure sore according to the invented method, just before the body is rested on a mattress.

FIG. 10 is a sectional view (not to scale) of a portion of a human body, which body has a pressure sore, resting on a portion of a mattress, with a cushion disposed against the pressure sore according to the invented method.

FIG. 11 is a side elevational view of a patient rolled over on to the patient's side while lying on a mattress. The patient is shown having a pressure sore, shown in dashed lines, by the patient's coccyx, with a rectangular cushion used in the invented method applied in contact with the pressure sore.

FIG. 12 is an enlarged side elevational view of the rectangular cushion shown in FIG. 11.

FIG. 13 is an enlarged top plan view of the rectangular cushion shown in FIG. 11.

FIG. 14 is a sectional view (not to scale) of a portion of a human body, which body has a pressure sore, with a dressing applied to the pressure sore and a cushion disposed against the dressing according to the invented method, just before the body is rested on a mattress.

FIG. 15 is a sectional view (not to scale) of a portion of a human body, which body has a pressure sore, with a dressing applied to the pressure sore and a cushion disposed against the dressing according to the invented method.

FIG. 16 is a top plan view of a cushion in a harness.

### DETAILED DESCRIPTION OF THE INVENTION

The preferred embodiments of the invention are described herein, with the benefit of the illustrations the attached drawings which are referred to herein. The same reference numeral will be used to identify identical elements throughout the drawings.

When a person is found to have a pressure sore, or complains of pain at an area of bony prominence or a nerve ending, it is preferred that the pressure sore or the area of pain, and the area around it, be cleaned and any dead tissue should be gently removed. For purposes of convenience, the term “pressure sore area” is used herein to refer to both a pressure sore and to an area of pain that may lead to a pressure sore.

Preferably after the pressure sore area has been cleaned, the invented steps should be performed.

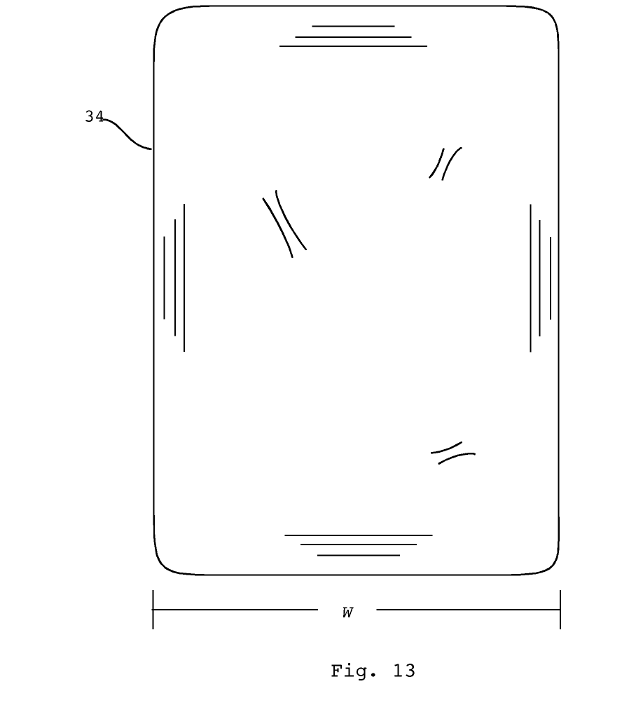

That is, an appropriately shaped and sized cushion, such as cushion 4, 24 or 34 shown in FIGS. 2, 3-7, and 9-16, should be positioned in direct contact, preferably substantially occlusive relationship, with the pressure sore area, and the skin immediately adjacent the pressure sore area. It is often preferred that prior to putting the cushion in position, a dressing such as 3M's Tegaderm brand transparent dressing be put in occlusive contact with the pressure sore area, and then the cushion is put in indirect contact with the pressure sore area by positioning the cushion adjacent the occlusive dressing. As much as practicable, the cushion should be maintained in that relationship with the pressure sore area and the skin immediately adjacent it when the patient is in his or her normal resting position.

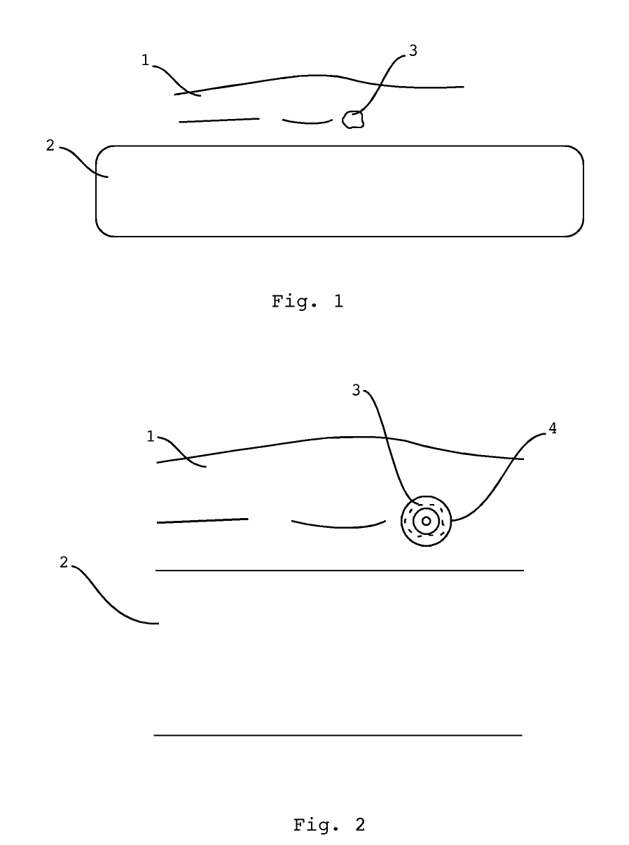

As shown in FIG. 1, a patient 1, resting on mattress 2, has been rolled onto her side. In this position, the patient's pressure sore 3, near her coccyx, can be seen. FIG. 8, which is a sectional view (not to scale) of a portion of a human body 1 resting on a mattress 2, illustrates an early stage pressure sore 3 in which the epidermis 15 and dermis 14 have been damaged, while the subcutaneous fat layer 13, muscle 12 and bone 11 have not. (While an early stage pressure sore is shown for purposes of describing the invention, the invention is intended for use as well in connection with late stage pressure sores.)

In FIG. 2, which is an enlarged view of a portion of the patient 1 and mattress shown in FIG. 1, the cushion 4 used in the invented method is shown in position against the pressure sore 3 (shown in dashed lines). In this view, the bottom of the cushion is seen.

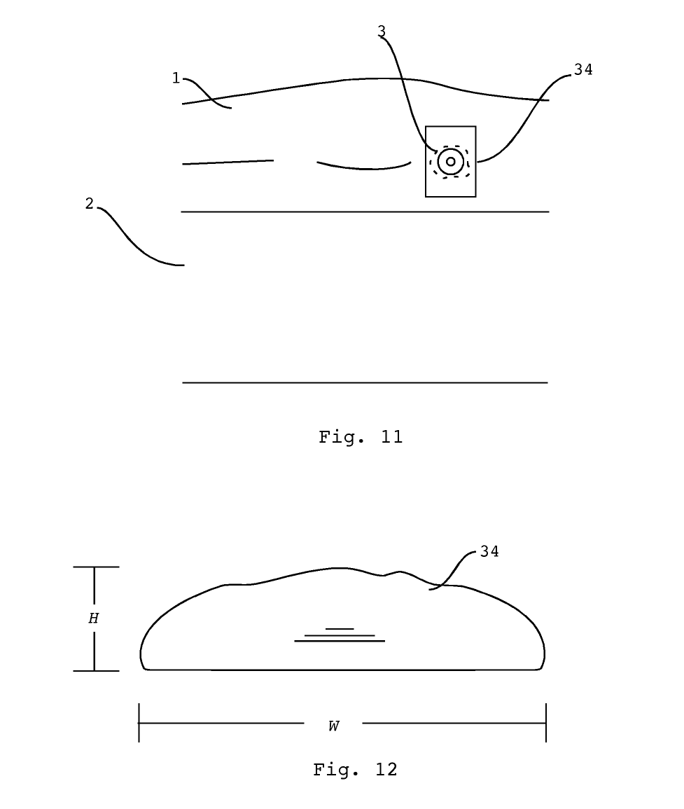

The cushion would typically be put in position by a caregiver, although some patients would have the ability to put the cushion in position themselves. With the cushion held in place, the patient is rolled back into normal resting position (i.e., supine). As a result, as illustrated in FIGS. 9 and 10, the cushion remains in a substantially occlusive contact with the pressure sore and surrounding area, or in contact with the dressing which is occlusively applied to the pressure sore and the surrounding area.

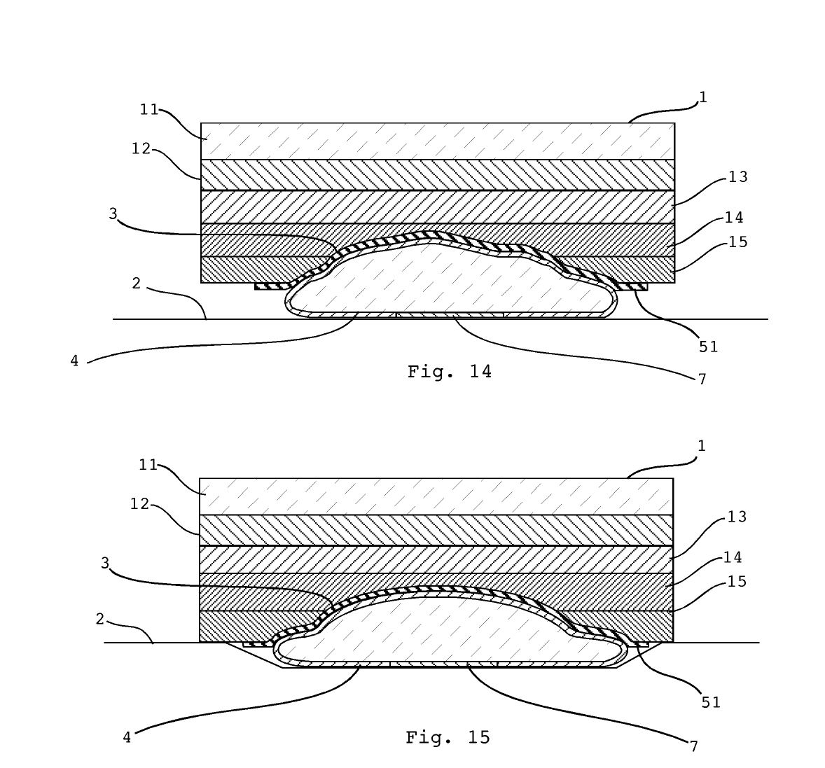

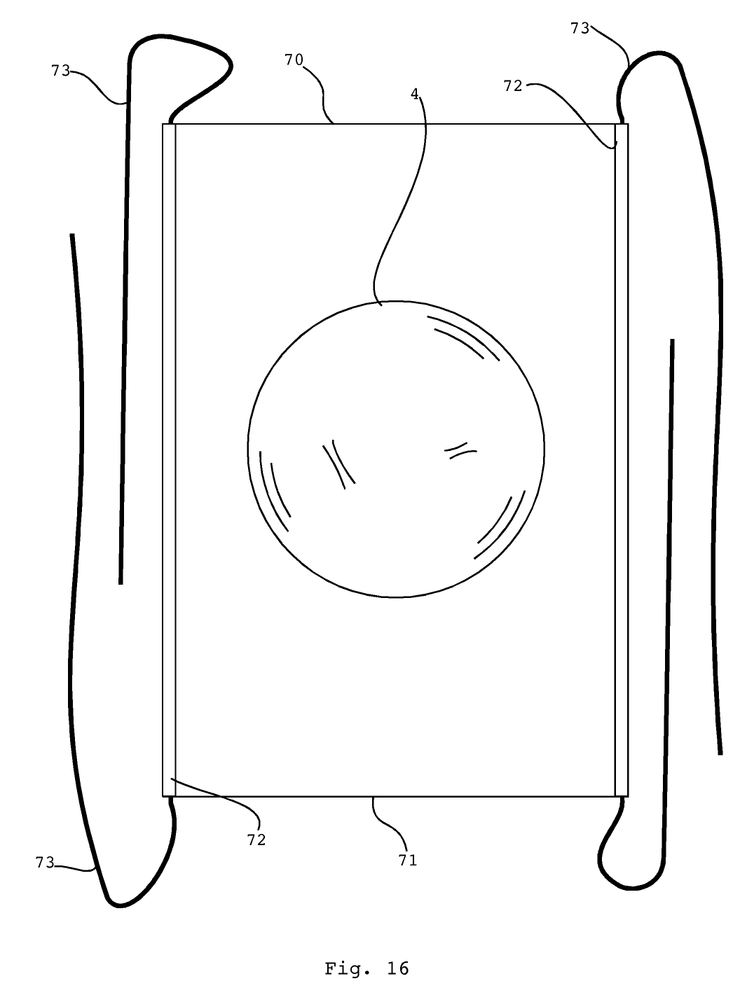

As shown in FIGS. 14 and 15, a dressing 51 may be placed in occlusive relationship with the pressure sore area and the surrounding area, with a cushion (cushion 4 in FIGS. 14 and 15), placed adjacent the dressing. If the cushion is held against the pressure sore area (or against the dressing if a dressing is used) even with slight pressure, a portion of the upper surface of the cushion will conform to the shape of the lesion formed by the pressure sore area as shown in FIG. 9 (and FIG. 14). When the cushion is held against the lesion (or dressing) with great pressure, or under the pressure of a patient's weight while lying on a mattress, with the cushion's bottom resting against the mattress 2 (or against whatever surface the patient is resting upon), the upper surface of the cushion will tend to form into a rounded shape and the lesion (with any dressing in occlusive relationship with it) in contact with the cushion would tend to conform to that shape, particularly when the pressure sore is in an early stage as shown in FIG. 10 (and FIG. 15). (While no illustration is to scale, FIGS. 8, 9, 10, 14 and 15 are particularly out of scale.) When the cushion is used with later stage pressure sores, part of the upper surface of the cushion would at least somewhat tend to conform to the shape of the lesion.

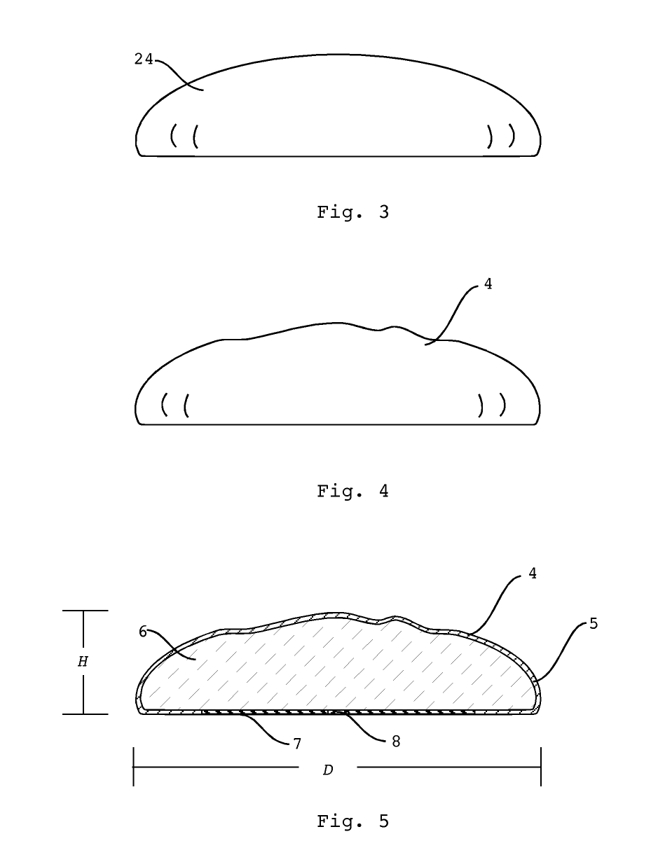

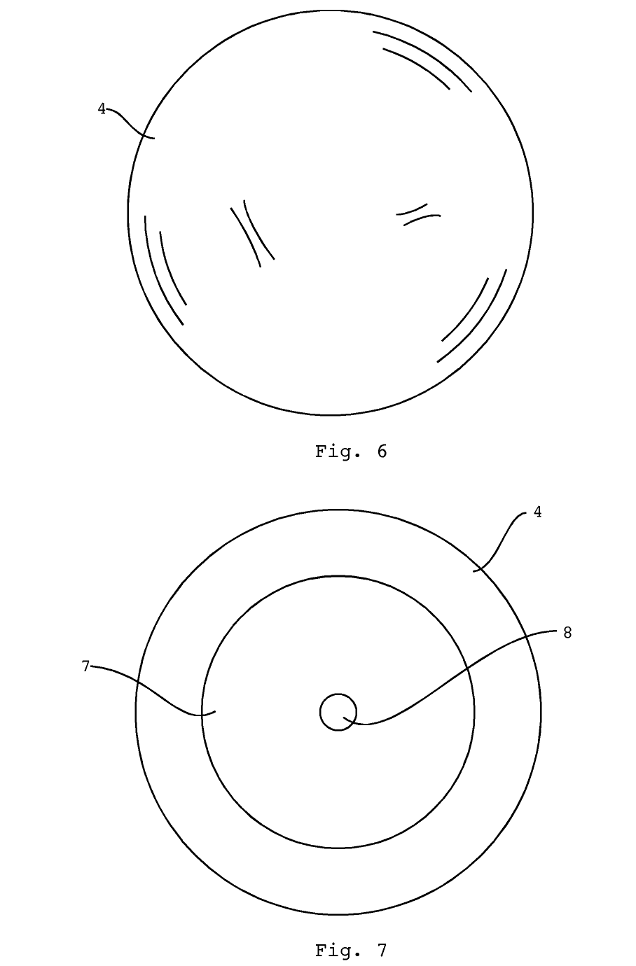

A cushion used in the invented method is illustrated in FIGS. 3-7. As shown in FIG. 5, cushion 4 has a shell 5 and a fluid fill 6 that may be a liquid, for example, water or saline water, or a gel. The currently preferred fill is silicone gel, such as the silicone fill used in breast implants. The shell would typically include a fill hole 8 along its bottom surface, and it would typically also include a bottom patch 7 surrounding fill hole 8. A gaseous fill would work, but not as well as a liquid or gel fill. (For purposes of the description herein, a fluid may be a gas or a liquid, and a gel which is easily deformable is considered a liquid.)

Shell 5 is formed of a flexible material, preferably an elastomeric material. The material used should be formed so that it is substantially impermeable to fluids. Polyurethane or rubber could be used to form the shell. The most preferred material is elastomeric silicone. The outer surface of the shell may be textured or smooth. In the preferred embodiment the outer surface is smooth. The goal is to reduce as much as possible the friction which would be created between the shell and the skin of the patient when the cushion is in actual use.

In the preferred embodiment, the shell is filled substantially completely with liquid and/or gel, with no gaseous substance (for example, air) being present within the shell, or as little gaseous substance as practicable being present.

The shell could be filled to its capacity, such as the case with cushion 24 shown in FIG. 3. However, in the preferred embodiment, shown in FIGS. 4-7, the shell of cushion 4 is only partially filled so that the shell has slack in it.

In the preferred embodiment of the invention, the cushion is selected for a particular pressure sore such that the perimeter of the cushion extends slightly beyond the perimeter of the lesion, as shown in FIGS. 2, 9 and 10.

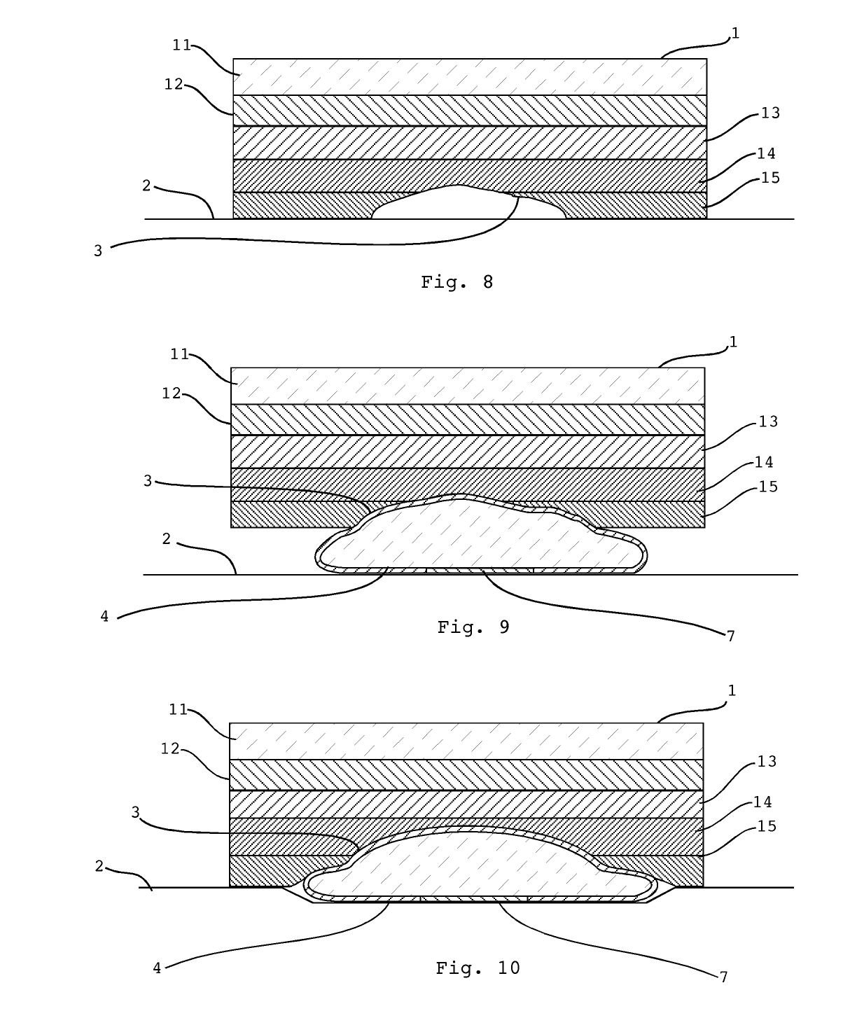

As shown in FIGS. 6 and 7, the cushion, when viewed from above with its bottom surface resting on a flat surface, is substantially circular in shape. This is the preferred shape. However, as shown in FIGS. 11, 12 and 13, a cushion 34 having a rectangular (including square), or other shape may be used in the invented method.

When the cushion is circular, its diameter D when it is placed on a flat surface, with no external pressure applied, is between about 1.5 and about 6 times its height H as shown in FIG. 5. Preferably, the cushion's diameter is between 3 and 4 times its height. If the cushion has a different shape, as shown, for example, in FIG. 12, its width W when it is laid upon a flat horizontal surface, with no external pressure applied, should be between about 1.5 and about 6 times its height H, and preferably between 3 and 4 times its height.

Those in the medical field will recognize the cushion used in the invented method as being the same as, or similar to, breast implants. It follows that makers of breast implants would have the know-how to make the cushions described herein.

In connection with the invented method, the cushion will transmit significant pressure against the pressure sore area.

While this is contrary to the typical recommendation that pressure be removed from the pressure sore as much as possible, this application of positive pressure through the cushion to the pressure sore area is an intended result of the invented method.

In addition, the specific heat index of the cushion is relatively high. As a result, a cushion at room temperature can be placed against a patient without causing a feeling of chill, yet for between one and three hours the cushion would draw heat away from the pressure sore area. This is an intended result of the invented method and in this regard treatment with the cushion is consistent with the typical recommendation that heat be removed from a pressure sore. When a cushion is first put into position, its temperature should be less than 96° F., and it is preferred that its temperature be room temperature or less.

To the extent that the patient makes small shifts relative to the mattress, friction is almost completely eliminated at the pressure sore area and the area immediately around it. That is because almost all the movement, and any friction associated with it, relative to the mattress is absorbed by the cushion, and the upper surface of the cushion would not move much, if at all, relative to the pressure sore area (or relative to a dressing if a dressing is used). To the extent, if any, that the pressure sore area does move relative to the upper surface of the cushion, the friction between the pressure sore area (or a dressing) and the upper surface of the cushion is very low.

Another advantage provided by the use of the invented method relates to a patient lying supine on a mattress. Using the invented method, the pressure sore area is lifted above the surface of the mattress, substantially reducing the risk that the pressure sore area would be contaminated by the results of any bowel and/or urinary incontinence.

After about an hour or two the cushion should be replaced with another cushion. The replacement cushion should be about room temperature. However, the replacement cushion could be cooled beforehand, which may provide further relief from discomfort for the patient over a longer period of time. Of course, in the case of a cooled cushion, the patient would be warned to expect an “initial contact” chill.

When the cushion is being replaced, the patient may be turned. If a dressing (such as Tegaderm brand transparent dressing) is used, it may be changed as needed, with the pressure sore area cleaned as is done in typical basic care. In addition, antibiotics or other salves may be administered to the pressure sore area.

Before positioning a cushion against a pressure sore area (or against a dressing in occlusive relationship with the pressure sore area), the cushion should be cleaned with a mild soap or an antibacterial soap, and fully rinsed and dried with a, preferably, lint free cloth.

When a pressure sore area is on the part of a body vertically above the resting surface (e.g., a pressure sore near a patient's coccyx when the patient lies supine), the pressure exerted through the cushion against the lesion actually comes from the body weight. For pressure sore areas located where body weight cannot provide the pressure, or for applying a cushion to pressure sore areas on particular parts of a body which might roll off of a cushion, or for accommodation of a patient in a wheel chair, a cushion may be held in position against a pressure sore area with a bandage, strap, belt, wrap or other device. Such a device will be referred to as a “harness” herein. In addition, the bottom surface of the cushion (i.e., the portion of the cushion that would face away from the patient (i.e., the portion with the fill hole)), may be fitted with fastening material which would help hold the cushion in place on a surface.

For example, for a pressure sore area located at a patient's heel bone, a cushion could be held in place with the harness illustrated in FIG. 16. The illustrated harness 70 includes a rectangular-shaped cloth 71 made of breathable cotton with draw string channels 72 formed along each of its two long edges. A draw string 73 is positioned in each channel, with the draw string's ends exposed. A cushion, such as cushion 4, is positioned approximately in the center of the cloth. The cushion, slung in the harness, is then positioned against the pressure sore area (or against the dressing applied to the pressure sore area), with the draw stings tied over the patient foot's instep and around the leg's ankle. The cushion is thus maintained in pressure relationship with the pressure sore area (i.e, pressure is applied to the pressure sore area).

Other examples of harnesses, such as an elastic bandage wrapped around the foot and lower portion of the leg, would be obvious to those skilled in the medical art.

The method described herein for treating pressure sores can also be used to prevent them by the occasional use of a cushion at any spot where a pressure sore is expected to occur.

By way of summary, the invented method for treating or preventing pressure sores comprises (i.e., includes but is not limited to) the following steps: applying a cushion in occlusive contact with substantially the entire surface of a pressure sore area, and applying pressure on the pressure sore area through the cushion. The cushion is preferably circular in shape (although it may have other shapes) with its diameter between about 1.5 and about 6 times its height, and preferably between about 3 and about 4 times its height. In the preferred embodiment, the cushion has a silicone elastomeric shell, with the portion of the shell which is intended to be in occlusive contact with the pressure sore area having a smooth outer surface. In the preferred embodiment, the cushion is only partially filled, and the fill material is silicone gel. The cushion may be held in place by means of a harness. Further, rather than having the cushion in direct contact with the pressure sore area, a dressing may be put in direct, preferably occlusive, contact with the pressure sore area, and the cushion would be disposed in contact with the dressing. In other words, the cushion would be in indirect contact with the pressure sore area. Pressure would be applied against the pressure sore area through the cushion and dressing.

It will be understood that various changes of the details, materials, steps, arrangement of steps and uses which have been herein described and illustrated in order to explain the nature of the invention will occur to and may be made by those skilled in the art, and such changes are intended to be included within the scope of this invention.

I claim the following:

1. A method for treating or preventing pressure sores, said method comprising the following steps:
applying a cushion in contact with substantially the entire surface of a pressure sore area, and
applying pressure against the pressure sore area through the cushion.

2. The method of claim 1, wherein said cushion is comprised of a flexible shell filled with a fill material which is a fluid.

3. The method of claim 1, wherein said cushion is comprised of a flexible shell formed of an elastomeric material and said shell is only partially filled with a fill material, which fill material is a fluid, so that slack remains in the shell.

4. The method of claim 3, wherein said shell is formed of elastomeric silicone.

5. The method of claim 4, wherein said fill material is a liquid.

6. The method of claim 5, wherein said liquid is comprises water.

7. The method of claim 5, wherein said liquid comprises saline water.

8. The method of claim 5, wherein said liquid comprises silicone gel.

9. The method of claim 5, wherein said cushion, when laid upon a flat, horizontal surface, has a substantially circular shape when viewed from above, and said cushion's diameter is between about 3 and 4 times its height.

10. The method of claim 5, wherein said cushion, when laid upon a flat, horizontal surface, has a substantially circular shape when viewed from above, and said cushion's diameter is between about 1.5 and 6 times its height.

11. The method of claim 5, wherein said cushion, when laid upon a flat, horizontal surface, has a substantially rectangular shape when viewed from above, and said cushion's width is between about 3 and 4 times its height.

12. The method of claim 5, wherein said cushion, when laid upon a flat, horizontal surface, has a substantially rectangular shape when viewed from above, and said cushion's diameter is between about 1.5 and 6 times its height.

13. The method of claim 5, wherein said cushion has a portion of its shell which is intended to be positioned against said pressure sore area, said portion having a smooth outer surface.

14. The method of claim 5, wherein said cushion is held in place against said pressure sore area by means of a harness.

15. The method of claim 5, comprising the additional step of replacing said cushion with second cushion, which second cushion has a temperature of less than 96 degrees Fahrenheit.
